(19) United States
(12) Patent Application Publication
(10) Pub. No.: US 2014/0221834 A1
(43) Pub. Date: Aug. 7, 2014

(54) CATHETER INSERTION ASSIST DEVICE AND TREATMENT APPARATUS

(71) Applicant: OLYMPUS CORPORATION, Tokyo (JP)

(72) Inventor: Miyuki MURAKAMI, Tokyo (JP)

(73) Assignee: OLYMPUS CORPORATION, Tokyo (JP)

(21) Appl. No.: 14/253,112

(22) Filed: Apr. 15, 2014

Related U.S. Application Data
(63) Continuation of application No. PCT/JP2012/081591, filed on Nov. 29, 2012.

(30) Foreign Application Priority Data
Dec. 28, 2011 (JP) 2011-289878

(51) Int. Cl. A61M 37/00; A61N 7/02; A61M 25/01; A61B 19/00

(52) U.S. Cl. CPC A61M 37/0092; A61B 19/5244; A61N 7/02; A61M 25/01; A61B 2019/5276
USPC 600/439

(57) ABSTRACT

Operative duration is shortened, and strain on a patient is reduced. Provided is a catheter insertion assist device comprising an ultrasonic element arranged at a tip of an insertion portion to be inserted into the body, and emitting an ultrasonic wave toward outside the insertion portion; and a guide portion for guiding a catheter so as to protrude the catheter toward a region irradiated with an ultrasonic wave from the ultrasonic element.

### TECHNICAL FIELD

The present invention relates to a catheter insertion assist device and a treatment apparatus.

### BACKGROUND ART

In the case where arrhythmia treatment is performed by approaching a heart from outside the heart, fat adhered to the surface of the heart is an obstacle to treatment. Thus, a treatment device such as a catheter is not able to reach the surface of the cardiac muscle. Therefore, it is expected to remove fat adhered to the surface of the heart.

Conventionally, liposuction machines have been known, in which in order to remove fat from the surface of organs, a tip of a device is inserted into adipose tissue, a needle provided at the tip of the device is subjected to ultrasonic vibrations to generate frictional heat around the needle to melt fat, and physiological saline and the like is injected to emulsify the fat which is sucked and removed (for example, see Patent Literature 1).

### CITATION LIST

### Patent Literature

### {PTL 1}

Japanese Examined Patent Application, Publication No. Hei 6-20462

### SUMMARY OF INVENTION

### Technical Problem

In the case of use of the liposuction machine in Patent Literature 1, removal of fat and treatment are needed to be performed sequentially. In other words, it is required that the liposuction machine is inserted into the body to remove fat, and then the liposuction machine is removed from the body, and thereafter a catheter for arrhythmia treatment is inserted into the body to perform treatment. Therefore, an operation contains many procedures, is complicated, is time-consuming, and puts great strain on a patient.

The present invention provides a catheter insertion assist device and a treatment apparatus capable of shortening operative duration, and reducing strain on a patient.

### Solution to Problem

A first aspect of the present invention provides a catheter insertion assist device comprising an ultrasonic element arranged at a tip of an insertion portion to be inserted into a body, and emitting an ultrasonic wave toward outside the insertion portion, and a guide portion for guiding a catheter so as to protrude the catheter toward a region irradiated with an ultrasonic wave from the ultrasonic element.

In the above aspect, a stopper for restricting the amount of protrusion of the catheter may be provided in the guide portion.

In the first aspect, an opening allowing passage of the catheter may be provided in the ultrasonic element.

In the first aspect, it may further comprise sealing means for sealing in a liquid-tight manner a space between the ultrasonic element and an affected area including the irradiated region; and liquid supplying means for filling the space with a liquid.

In the configuration, liquid circulating means for circulating the liquid in the space may be included.

A second aspect of the present invention provides a catheter insertion assist device comprising a cap detachably/attachably attached to a tip of an endoscope insertion portion having a channel for introducing a catheter, the cap having an aperture at one end, and an ultrasonic element arranged in the cap, introduced to the tip of the endoscope insertion portion via the channel, and emitting an ultrasonic wave toward the proximity of a protrusion position of the catheter protruded outside from the cap through the aperture.

In the second aspect, the device may further comprise sealing means arranged around the entire perimeter of the aperture of the cap, and for sealing in a liquid-tight manner a space between the ultrasonic element and an affected area including a region irradiated with the ultrasonic wave from the ultrasonic element, and liquid supplying means for filling the space with a liquid.

In the above configuration, liquid circulating means for circulating a liquid in the space may be included.

In the first aspect and the second aspect, the device may further comprise monitoring means for monitoring a protrusion of the catheter.

Moreover, in the above configuration, the monitoring means may be a monitoring ultrasonic element acquiring an ultrasonic tomogram in the proximity of the protrusion position of the catheter.

A third aspect of the present invention provides a treatment apparatus comprising any one of the above catheter insertion assist devices and a catheter, wherein a potential detecting electrode is provided which detects a potential of body tissue that is in contact with a tip of the catheter.

### BRIEF DESCRIPTION OF DRAWINGS

FIG. 1 is the whole configuration view showing a treatment apparatus comprising a catheter insertion assist device according of one embodiment of the present invention.

FIG. 2A is a vertical cross-sectional view showing the catheter insertion assist device in FIG. 1, and showing a state of adipose tissue being sucked.

FIG. 2B is a vertical cross-sectional view showing the catheter insertion assist device in FIG. 1, and showing a state in which the adipose tissue condition is measured before emitting an ultrasonic wave.

FIG. 2C is a vertical cross-sectional view showing the catheter insertion assist device in FIG. 1, and showing a state of the adipose tissue is irradiated with the ultrasonic wave.

FIG. 3A is a vertical cross-sectional view showing the catheter insertion assist device in FIG. 1, and showing a state in which an adipose tissue condition is measured after emitting the ultrasonic wave.

FIG. 3B is a vertical cross-sectional view showing the catheter insertion assist device in FIG. 1, and showing a state of the catheter being inserted into a softened adipose tissue.

FIG. 4A is a view showing an ultrasonic waveform in measuring in FIG. 2B.

FIG. 4B is a view showing an ultrasonic waveform in measuring in FIG. 3A.

FIG. 5 is a vertical cross-sectional view showing a first modification of the catheter insertion assist device in FIG. 2.

FIG. 6 is a vertical cross-sectional view showing a second modification of the catheter insertion assist device in FIG. 2.

FIG. 7 is a vertical cross-sectional view showing a third modification of the catheter insertion assist device in FIG. 2.

FIG. 8 is a vertical cross-sectional view showing a fourth modification of the catheter insertion assist device in FIG. 2.

### DESCRIPTION OF EMBODIMENTS

A catheter insertion assist device 1 according to one embodiment of the present invention will be described below with reference to drawings.

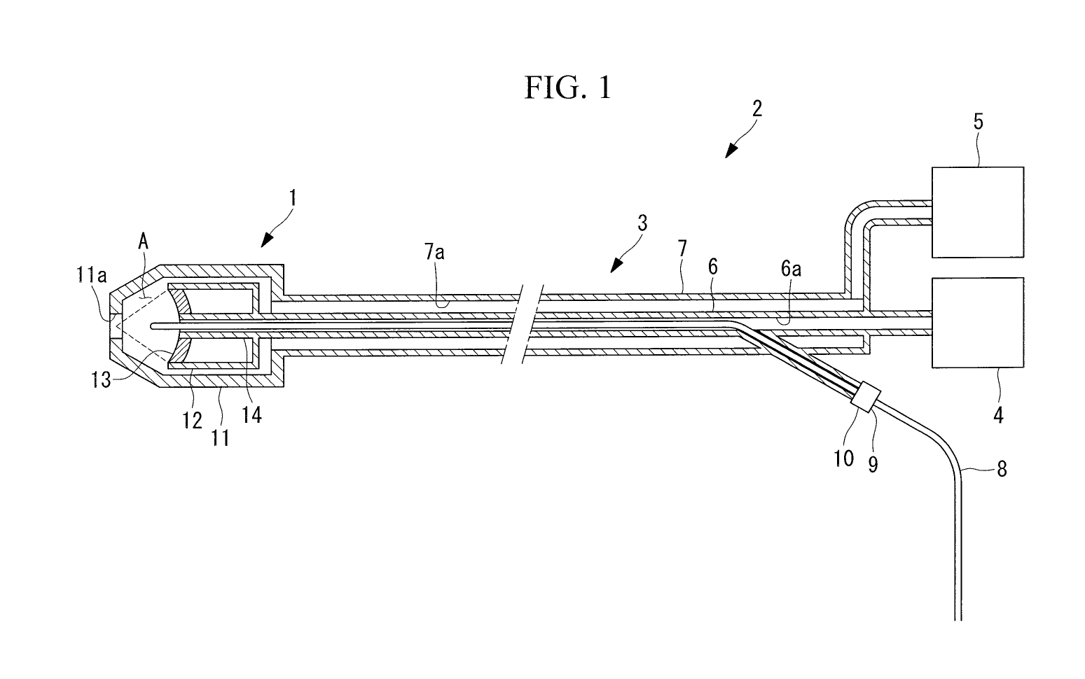

As shown in FIG. 1, the catheter insertion assist device 1 according to the embodiment is included in a treatment apparatus 2. The treatment apparatus 2 includes an insertion portion 3 to be inserted into the body, the catheter insertion assist device 1 according to the embodiment attached to a tip of the insertion portion 3, a feed pump 4 and a drainage pump 5 connected to a base end of the insertion portion 3, and a catheter 8 described below.

The insertion portion 3 has a double tube structure including an inner conduit 6 having a channel 6a passing through a central region in a radial direction along a longitudinal direction, and an outer conduit 7 having an outer channel 7a arranged outside the inner conduit 6. The inner conduit 6 is connected on the base end side thereof to the feed pump (liquid supplying means, liquid circulating means) 4, and the outer conduit 7 is connected on the base end side thereof to the drainage pump (liquid circulating means) 5.

The insertion portion 3 further includes, on the base end side thereof, an inlet 9 through which a catheter 8 passes through the outer conduit 7 in a radial direction and inserted into the inner conduit 6. The inlet 9 is provided with a seal member 10 that supports the catheter 8 movably in the longitudinal direction and seals the periphery of the catheter 8 in a liquid-tight manner. The catheter 8 is an ablation catheter having an electrode (not shown) at a tip thereof, and cauterizes body tissue that is in contact with the tip depending on energization, as an example.

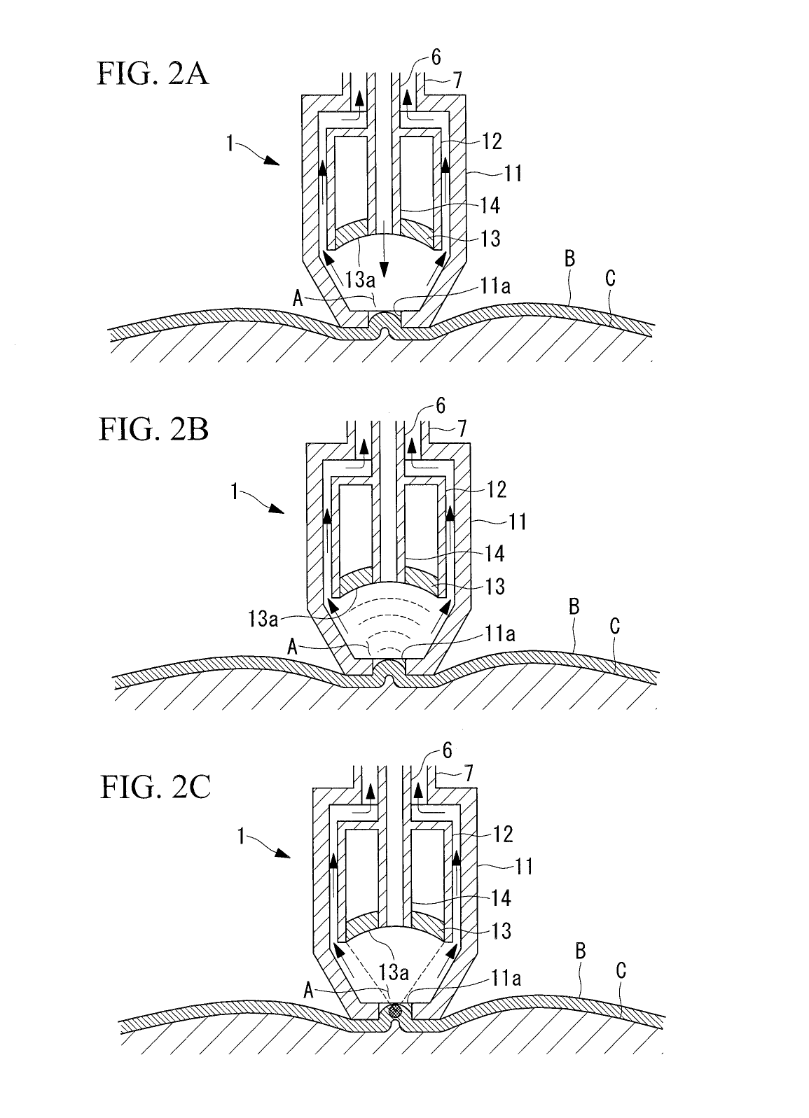

As shown in FIG. 2, the catheter insertion assist device 1 according to the embodiment includes a substantially cylindrical shape housing (cap) 11 having an opening 11a at a tip thereof, a first inner cylindrical member 12 arranged in the housing 11, an ultrasonic element 13 arranged at a tip of the first inner cylindrical member 12, and a second inner cylindrical member (a guide portion) 14 provided through the center of the ultrasonic element 13.

The housing 11 is tapered in the tip thereof. Moreover, the inside of the housing 11 located inside the opening 11a defines a space A in front of the ultrasonic element 13.

The housing 11 is, for example, made of an optically transparent material such that an internal state may be checked from outside with, e.g., an endoscope.

A circular tube-shaped flow passage between the housing 11 and the first inner cylindrical member 12 is in communication with the space A located at the tip of the housing 11 and the outer conduit 7 of the insertion portion 3, and the inside of the second inner cylindrical member 14 is in communication with the space A located at the tip of the housing 11 and the inner conduit 6 of the insertion portion 3.

Thus, a transparent ultrasonic wave conducting medium (for example, physiological saline) supplied by the feed pump 4 is introduced into the space A located at the tip of the housing 11 via the inner conduit 6 and the flow passage in the second inner cylindrical member 14 to fill the space A. Moreover, by activating a drainage pump 5, the ultrasonic wave conducting medium in the space A located at the tip of the housing 11 is sucked via the flow passage between the housing 11 and the first inner cylindrical member 12, and via the outer conduit 7 so as to reduce the pressure of the space A.

The ultrasonic element 13 has a substantially concave spherical ejection face 13a. Ultrasonic waves ejected from the substantially concave spherical ejection face 13a are to be focused into its focal position. As shown in FIG. 1, the focus of the ejection face 13a is positioned at a location corresponding to the opening 11a of the housing 11.

The ultrasonic element 13 is arranged so as to enclose the tip of the first inner cylindrical member 12, and thereby the back side of the ejection face 13a is in contact with air. Therefore, the ultrasonic element 13 does not emit an ultrasonic wave to the back side, and is adapted to be capable of efficiently ejecting an ultrasonic wave only from the ejection face 13a side.

An operation of the catheter insertion assist device 1 according to the embodiment as configured in this way will be described below.

In order to bring the catheter 8 into the proximity of the surface of body tissue C such as cardiac muscular tissue positioned in a layer under adipose tissue B using the catheter insertion assist device 1 according to the embodiment, as shown in FIG. 2A, the insertion portion 3 having the catheter insertion assist device 1 arranged at the tip is inserted into the body of a patient, and the tip face of the housing 11 of the catheter insertion assist device 1 is brought into intimate contact with the surface of the adipose tissue B.

The opening 11a is formed in the tip face of the housing 11, and thus the opening 11a is placed so as to be sealed by the surface of the adipose tissue B. Since this allows the opening 11a of the housing 11 to be sealed by the adipose tissue B, as shown in FIG. 2A, the feed pump 4 and the drainage pump 5 are activated to supply an ultrasonic wave propagation medium into the housing 11 via the inner conduit 6 to fill the space A in the housing 11, and the drainage pump 5 is used to reduce the pressure of the space A in the housing 11.

When the pressure of the space A in the housing 11 is reduced, the adipose tissue B which is in intimate contact with the opening 11a is sucked so as to be drawn into the housing 11. Then, the feed pump 4 is stopped, and the space A is maintained in a reduced pressure condition.

In this situation, the ultrasonic element 13 is activated. In other words, the space A in the housing 11 between the ultrasonic element 13 and the adipose tissue B is filled with the ultrasonic wave propagation medium, and therefore the ultrasonic wave emitted from the ultrasonic element 13 is efficiently propagated to the adipose tissue B.

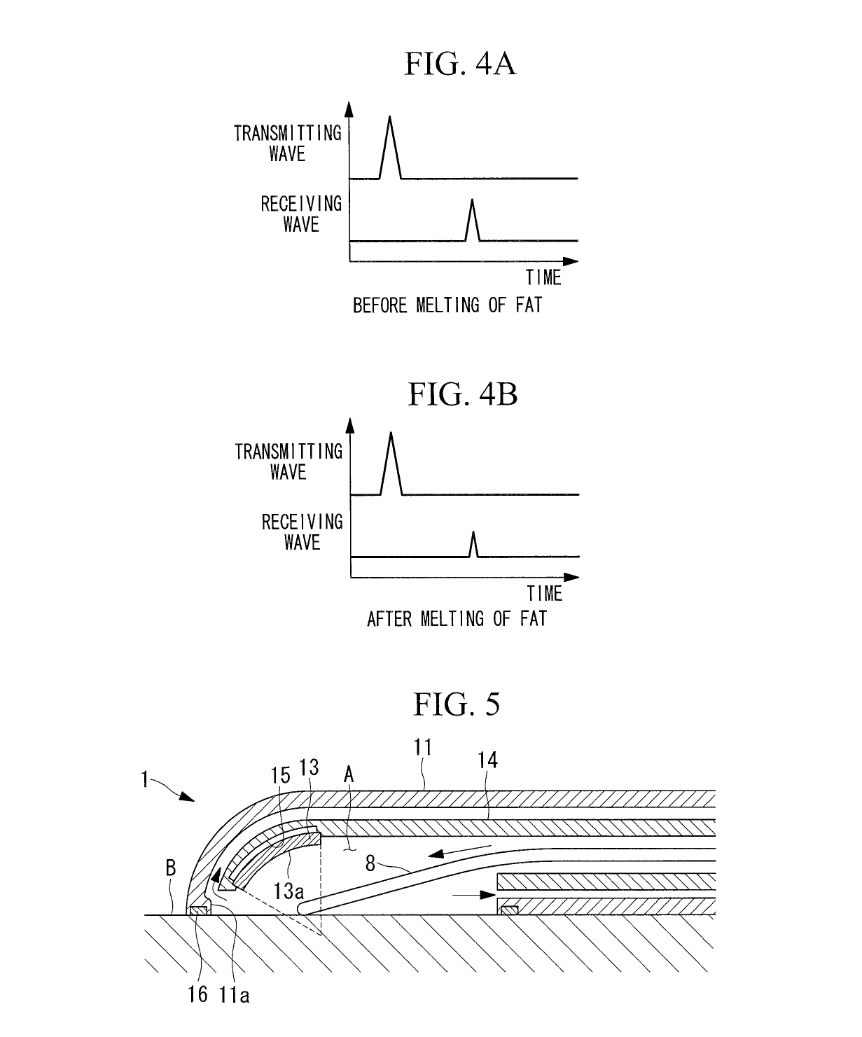

As shown in FIG. 2B, a single pulsed ultrasonic wave as shown in FIG. 4A is ejected, and the ultrasonic wave reflected back from the adipose tissue B is received. Then, the intensity of the received ultrasonic wave is stored at this time.

Next, as shown in FIG. 2C, a high-intensity ultrasonic wave having the central frequency of about 1 MHz is ejected from the ultrasonic element 13.

This allows the adipose tissue B arranged in a focused position of the ultrasonic wave in a sucked manner to be softened by heat caused by the ultrasonic wave.

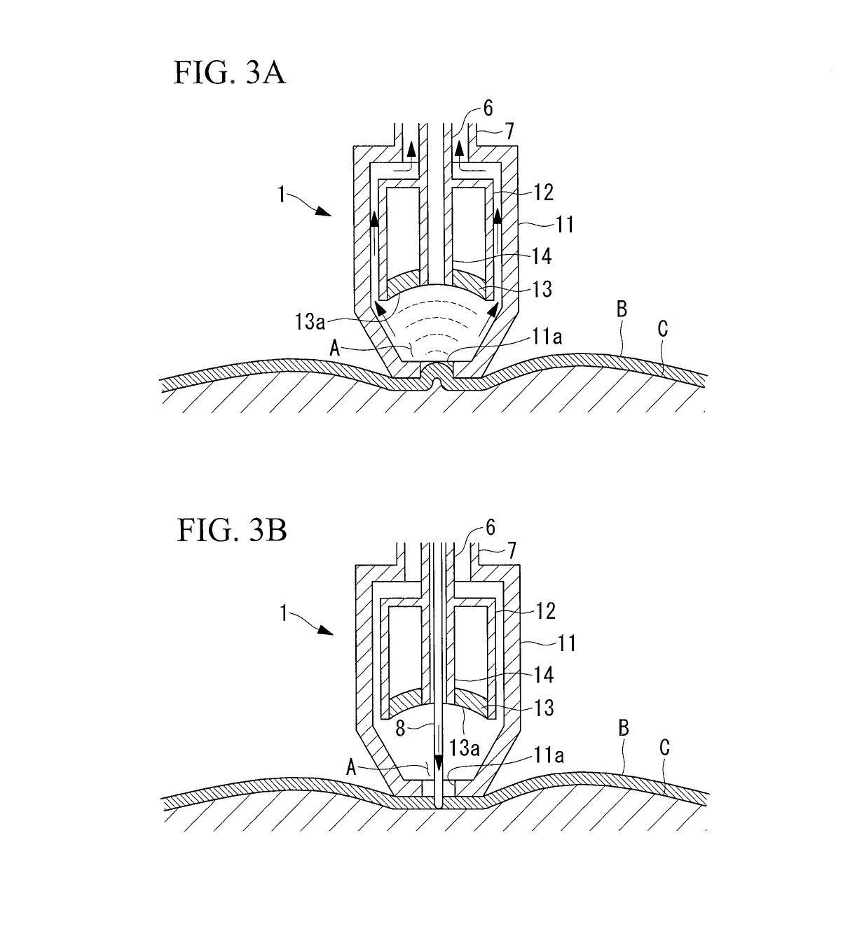

After this, as shown in FIG. 3A, a single pulsed ultrasonic wave as shown in FIG. 4B is ejected. The conditions such as the intensity of the pulse of the ejected ultrasonic wave are the same as the ultrasonic wave in FIG. 4A. Then, the ultrasonic wave reflected back from the softened adipose tissue B is received. At this time, if the intensity of the received ultrasonic wave is smaller by a predetermined rate than the stored intensity of the ultrasonic wave, it is possible to determine an achievement of sufficient softening of the adipose tissue B.

If it is determined that the adipose tissue B is sufficiently softened, as shown in FIG. 3B, the catheter 8 is introduced through the inner conduit 6, and the tip of the catheter 8 is pushed out from the center of the ultrasonic element 13 toward the opening 11a of the housing 11 via the second inner cylindrical member 14. In this case, since the adipose tissue B placed in the opening 11a is softened by the ultrasonic wave, the tip can be easily inserted into the adipose tissue B even if the catheter 8 has low stiffness and insufficient solidity.

The tip of the catheter 8 inserted into the adipose tissue B can be brought into the proximity of the surface of the body tissue C located in a layer under the adipose tissue B. The body tissue C may be cauterized depending on energization.

In other words, in accordance with the catheter insertion assist device 1 according to the embodiment, the catheter 8 is guided so as to be inserted into an irradiated region in the softened adipose tissue B irradiated with an ultrasonic wave. Therefore, compared with conventional methods in which a fat melting device is removed from the body after melting fat, and then a catheter is inserted, there are advantages in which troublesome work is unnecessary, operative duration can be shortened, and strain on a patient can be reduced.

It is noted that, in the embodiment, the catheter 8 is inserted after softening of the adipose tissue B by emitting an ultrasonic wave, and however, alternatively, the softening of the adipose tissue B by emitting an ultrasonic wave and the insertion of the catheter 8 may be simultaneously performed.

Moreover, a high-intensity ultrasonic wave having the central frequency of about 1 MHz is illustrated as an ultrasonic wave, and however, alternatively, an ultrasonic wave having the central frequency, several 10 kHz to several 10 MHz, may be used. In particular, in the case of improving an effect of melting the adipose tissue B by using ultrasonic wave absorption-based heating, an ultrasonic wave having a high frequency on the order of several 10 MHz, preferably, may be utilized to effectively utilize the absorption in the adipose tissue B. Moreover, in the case of improving an effect of melting the adipose tissue B based on fracture effect due to cavitation, an ultrasonic wave having a low frequency on the order of several 10 kHz, preferably, may be utilized to efficiently produce bubble growth. In this case, when bubble nuclei are contained in ultrasonic wave propagation medium, the cavitation can be efficiently generated at a low energy.

In the embodiment, an ultrasonic wave is emitted after the adipose tissue B is drawn into the opening 11a by reducing the pressure of the ultrasonic wave propagation medium with which the space A in the housing 11 is filled, and therefore there is an advantage in which a high-intensity convergent ultrasonic wave may not be emitted to the outside of the housing 11. Note that the reduction of the pressure in the ultrasonic wave propagation medium is not necessary and a portion of body tissue that is raised toward the inside of the opening 11a may be utilized by abutting the opening 11a of the housing 11 against a biological body.

In the embodiment, the concave spherical ultrasonic element of a single plate is illustrated as the ultrasonic element 13, and however, alternatively, a combination of divided plurality of ultrasonic elements 13 such as into a concentric pattern may be adopted. In the case of the combination of the plurality of ultrasonic elements 13, an ultrasonic wave may be converged in a desired position by utilizing a phase difference without adopting of a concave face shape. Thus, the focused position of the ultrasonic wave may be moved from the surface side of the adipose tissue B in a depth direction according to insertion of the catheter 8.

Referring to the ultrasonic wave, the combination of ultrasonic waves having a plurality of frequencies may be emitted. For example, it may be utilized that difference frequencies (difference tone) of 10 MHz, 25 MHz other than the basic frequency are effectively generated at a convergent position by simultaneously emitting ultrasonic waves having 5 MHz and 15 MHz. The combination of the plurality of frequencies preferably is set according to the thickness of the adipose tissue B.

In the embodiment, the ultrasonic element 13, which generates a convergent ultrasonic wave used for melting the adipose tissue B, transmits and receives a detecting pulsed ultrasonic wave to detect a state of adipose tissue before and after melting of the adipose tissue B, and however, alternatively, a molten state of the adipose tissue B may be checked through an additionally inserted endoscope by visual inspection. At this time, the cap is desirably a transparent member.

Moreover, an ultrasonic element (not shown) transmitting and receiving a ultrasonic wave used for detecting the state of the adipose tissue B may be provided, independently of the ultrasonic element 13 generating a convergent ultrasonic wave used for melting the adipose tissue B.

The convergence type ultrasonic element 13 having the substantially inner spherical ejection face 13a has been illustrated as the ultrasonic element 13, but the present invention is not limited thereto, and may adopt a plate-shaped ultrasonic element. In this way, emitting an ultrasonic wave over a relatively wide range allows the adipose tissue B to be softened.

In the embodiment, a through-hole is provided in the center of the ultrasonic element 13, and the catheter 8 is configured to advance and retreat. This allows the catheter 8 to be easily guided to an area irradiated with an ultrasonic wave. Alternatively, as shown in FIG. 5, the cylindrical shaped housing 11 has the opening 11a in a side thereof, wherein the ultrasonic element 13 emitting a convergent ultrasonic wave toward the opening 11a may be arranged in a position separate from the inner cylindrical member 14 for guiding the catheter 8.

A space 15 is provided on the back side of the ultrasonic element 13 such that an ultrasonic wave can be efficiently ejected to the ejection face 13a side.

Reference numeral 16 in the drawings denotes a seal member (sealing means), such as an O-ring or a gasket, arranged in a location surrounding the periphery of the opening 11a to seal a clearance between the housing 11 and the surface of the adipose tissue B.

Thus, the seal member 16 can seal the clearance between the housing 11 and the surface of the adipose tissue B so that the ultrasonic wave propagation medium can be stored in the space A in the housing 11. As a result, similarly to the above embodiment, the ultrasonic wave ejected from the ultrasonic element 13 efficiently propagates to the surface of the adipose tissue B, softening the adipose tissue B, and allowing for easy insertion of the catheter 8.

Moreover, according to the catheter insertion assist device 1 having the opening 11a in a side of the housing 11, there are advantages in which when the housing 11 is arranged such that the surface of the adipose tissue B encloses the opening 11a, the height dimension from the surface of the adipose tissue B is small since the housing 11 is arranged along the surface of the adipose tissue B. For example, in the case where, in a pericardial membrane, the surface of the cardiac muscle is treated, approach can be facilitated in a narrow space. Moreover, the ultrasonic element 13 has no through-hole for passage of the catheter 8, and therefore the effective area of the ultrasonic element 13 can be increased, and the intensity of the ultrasonic wave can be enhanced.

Although in the embodiment, there are provided the inner conduit 6 for supplying the ultrasonic wave propagation medium to the insertion portion 3 for insertion of the catheter 8, and the outer conduit 7 for sucking the ultrasonic wave propagation medium, the supplying and sucking of the ultrasonic wave propagation medium may be provided separately and independently of the insertion portion 3 for insertion of the catheter 8.

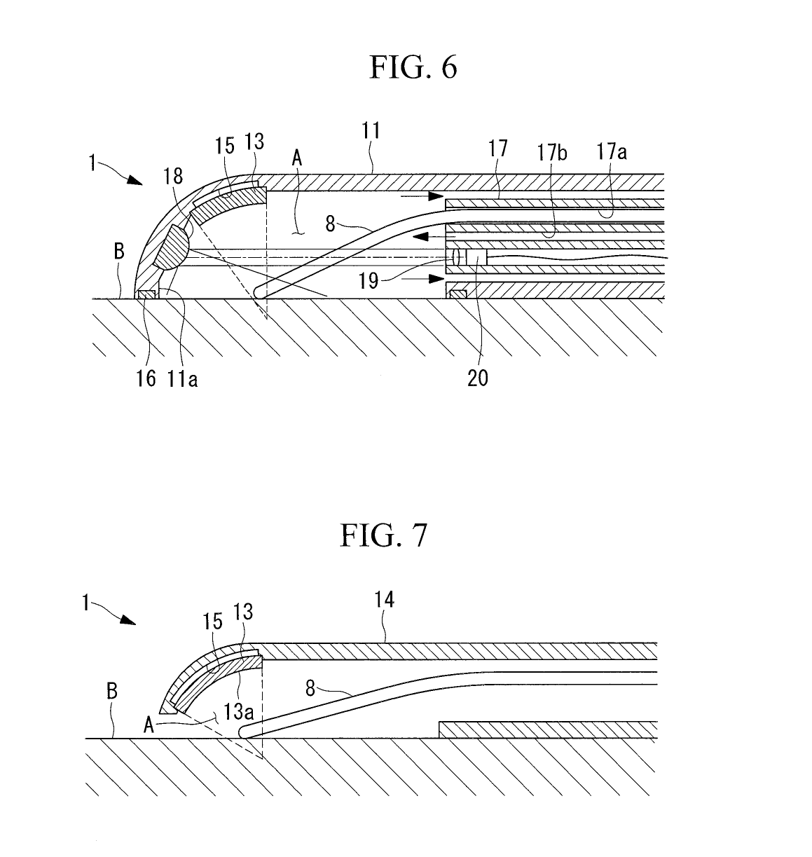

In the embodiment, the catheter insertion assist device 1 is attached to the tip of the double tube-shaped insertion portion 3, and however, alternatively, may be attached to a tip of an insertion portion 17 of the endoscope, as shown in FIG. 6.

In an example shown in FIG. 6, a mirror 18, which is arranged parallel with the ultrasonic element 13, is fixed to the housing (cap) 11. Light from a region irradiated with an ultrasonic wave is configured to enter an imaging device 20 through an objective lens 19 of the endoscope.

This allows softening of the adipose tissue B irradiated with the ultrasonic wave from the ultrasonic element 13 to be directly observed using the endoscope while insertion of the catheter 8 can be performed through a channel 17a. Moreover, the ultrasonic wave propagation medium can be supplied through the channel 17b included in the insertion portion 17.

Although, in the embodiment, the space A to be filled with the ultrasonic wave propagation medium is defined by the housing 11, alternatively, as shown in FIG. 7, in the case where a space around tissue treated by the catheter 8 is filled with the ultrasonic wave propagation medium, it is not necessary to provide the housing 11 defining the space.

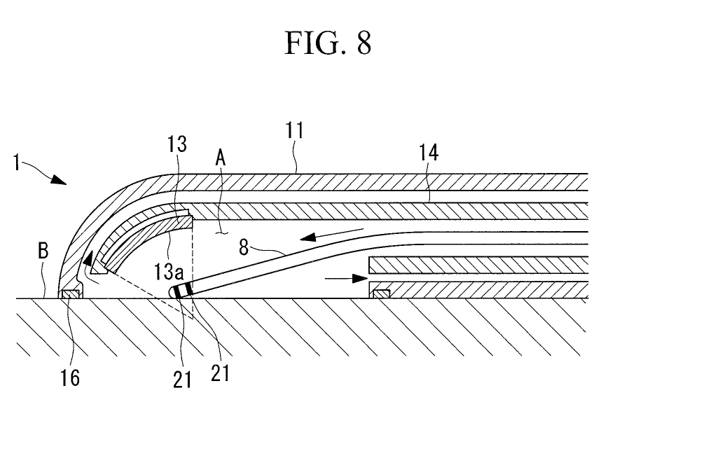

Moreover, as shown in FIG. 8, an electrode (potential detecting electrode, monitoring means) 21 for detecting a potential may be provided at the tip of the catheter 8.

This allows for monitoring whether the tip of the catheter 8 approaches body tissue to a distance at which the body tissue can be subjected to treatment or not since a potential detected by the electrode 21 increases as the electrode 21 approaches body tissue with the insertion of the catheter 8.

The electrodes 21 may be provided to face each other such that the focus is sandwiched between the electrodes 21 at a location in the proximity of the opening 11a, for example, the proximity of the seal member 16.

This allows the potential between two facing electrodes to be detected, and therefore it is possible to monitor whether abnormal site should be treated or not using the facing electrodes 21 by searching the position at which a detected abnormal signal is the maximum.

In order to prevent the catheter from excessively protruding, a stopper (not shown) may be provided between the second inner cylindrical member for guiding the catheter and the catheter so as to restrict protrusion of the catheter above a certain protrusion.

Moreover, the ultrasonic wave propagation medium may be circulated in the space A by balancing the supply amount of the ultrasonic wave propagation medium from the feed pump 4 and the suction amount from the drainage pump 5.

The following aspects can be derived from the embodiment described above.

A first aspect of the present invention provides a catheter insertion assist device comprising an ultrasonic element arranged at a tip of an insertion portion to be inserted into a body, and emitting an ultrasonic wave toward outside the insertion portion, and a guide portion for guiding a catheter so as to protrude the catheter toward a region irradiated with an ultrasonic wave from the ultrasonic element.

According to the first aspect, the insertion portion is inserted into the body when the catheter is introduced into body tissue, and the ultrasonic element arranged at the tip of the insertion portion is activated to generate the ultrasonic wave so that the body tissue is irradiated with the generated ultrasonic wave. In the case where the body tissue is covered with adipose tissue, the adipose tissue is melted and softened by emitted ultrasonic wave. In this situation, by guiding the catheter using the guide portion and protruding the catheter toward a region irradiated with the ultrasonic wave, the catheter can be easily entered into the softened adipose tissue.

In other words, even in the case where the tip of the catheter is not sharp-pointed or where the catheter has the flexibility and insufficient solidity, the catheter can be entered into the adipose tissue and be brought into the proximity of body tissue under adipose tissue. As a result, softening of fat and insertion of the catheter can be simultaneously advanced without complicated procedures such as insertion of the catheter after removal of fat firstly, operative duration can be shortened, and strain on a patient can be reduced.

In the above aspect, a stopper for restricting the amount of protrusion of the catheter may be provided in the guide portion.

This allows the amount of protrusion of the catheter to be restricted due to activating of the stopper, and the catheter to be stopped at the boundary between adipose tissue and body tissue. This can prevent the catheter from excessively pressing body tissue.

In the first aspect, an opening allowing passage of the catheter may be provided in the ultrasonic element.

This allows easy arrangement of the catheter in a region irradiated with the ultrasonic wave from the ultrasonic element by protruding the catheter from the opening formed in the ultrasonic element.

In the first aspect, it may further comprise sealing means for sealing in a liquid-tight manner a space between the ultrasonic element and an affected area including the irradiated region; and liquid supplying means for filling the space with a liquid.

In this way, the sealing means seals the space between the ultrasonic element and the affected area, and the liquid supplying means supplies the liquid to the space. Thus, the ultrasonic wave emitted from the ultrasonic element is conducted through the liquid to be efficiently emitted into the affected area.

In the configuration, liquid circulating means for circulating the liquid in the space may be included.

This allows the ultrasonic wave to be efficiently supplied to the affected area by activating the liquid circulating means to always supply a new liquid to the space even when the adipose tissue which is softened by the emitted ultrasonic wave from the ultrasonic element is released into the liquid.

A second aspect of the present invention provides a catheter insertion assist device comprising a cap detachably/attachably attached to a tip of an endoscope insertion portion having a channel for introducing a catheter, the cap having an aperture at one end, and an ultrasonic element arranged in the cap, introduced to the tip of the endoscope insertion portion via the channel, and emitting an ultrasonic wave toward the proximity of a protrusion position of the catheter protruded outside from the cap through the aperture.

According to the second aspect, the ultrasonic element arranged in the cap is activated with the cap attached to the tip of the endoscope insertion portion, and with the aperture of the cap faced to an affected area, the ultrasonic wave emitted from the ultrasonic element is emitted into the affected area through the aperture of the cap. This allows adipose tissue arranged in the affected area to be softened. In this situation, when the catheter is introduced through the channel of the endoscope insertion portion, the catheter is protruded in the proximity of an emitting position of the ultrasonic wave, and therefore the catheter can be easily entered into the softened adipose tissue.

In the second aspect, the device may further comprise sealing means arranged around the entire perimeter of the aperture of the cap, and for sealing in a liquid-tight manner a space between the ultrasonic element and an affected area including a region irradiated with the ultrasonic wave from the ultrasonic element, and liquid supplying means for filling the space with a liquid.

In this way, the aperture of the cap attached to the tip of the endoscope is sealed by the sealing means around the entire perimeter thereof, and the liquid is supplied into the space between the ultrasonic element and the affected area by the liquid supplying means. Thus, the ultrasonic wave emitted from the ultrasonic element is conducted through the liquid to be efficiently emitted into the affected area.

In the above configuration, liquid circulating means for circulating a liquid in the space may be included.

This allows the ultrasonic wave to be efficiently supplied to the affected area by activating the liquid circulating means to always supply a new liquid to the space even when the adipose tissue which is softened by the emitted ultrasonic wave from the ultrasonic element is released into the liquid.

In the first aspect and the second aspect, the device may further comprise monitoring means for monitoring a protrusion of the catheter.

This allows the catheter to be entered while the amount of protrusion of the catheter is monitored by the monitoring means.

Moreover, in the above configuration, the monitoring means may be a monitoring ultrasonic element acquiring an ultrasonic tomogram in the proximity of the protrusion position of the catheter.

This allows the catheter to be entered while the protrusion position of the catheter is monitored in the ultrasonic tomogram acquired by activation of the monitoring ultrasonic element.

A third aspect of the present invention provides a treatment apparatus comprising any one of the above catheter insertion assist devices and a catheter, wherein a potential detecting electrode is provided which detects a potential of body tissue that is in contact with a tip of the catheter.

According to the third aspect, the potential detecting electrode detects the potential of the body tissue that is in contact with the tip of the catheter, and this allows the state proximate to the body tissue to be monitored. In other words, in the case where the body tissue is covered with adipose tissue, the catheter is inserted into the adipose tissue melted and softened by the emitted ultrasonic wave, and a potential detected by the potential detecting electrode increases as the catheter approaches the body tissue, for example, cardiac muscular. This allows for monitoring whether the catheter approaches to a distance at which the body tissue can be subjected to treatment or not.

1. A catheter insertion assist device, comprising:
an ultrasonic element arranged at a tip of an insertion portion to be inserted into a body, and emitting an ultrasonic wave toward outside the insertion portion; and
a guide portion for guiding a catheter so as to protrude the catheter toward a region irradiated with an ultrasonic wave from the ultrasonic element.

2. The catheter insertion assist device according to claim 1, wherein a stopper for restricting the amount of protrusion of the catheter is provided in the guide portion.

3. The catheter insertion assist device according to claim 1, wherein an opening allowing passage of the catheter is formed in the ultrasonic element.

4. The catheter insertion assist device according to claim 1, further comprising:
sealing member for sealing in a liquid-tight manner a space between the ultrasonic element and an affected area including the irradiated region; and
liquid supplying device for filling the space with a liquid.

5. The catheter insertion assist device according to claim 4, further comprising: liquid circulating device for circulating the liquid in the space.

6. A catheter insertion assist device, comprising:
a cap detachably/attachably attached to a tip of an endoscope insertion portion having a channel for introducing a catheter, the cap having an aperture at one end; and
an ultrasonic element arranged in the cap, introduced to the tip of the endoscope insertion portion via the channel, and emitting an ultrasonic wave toward the proximity of a protrusion position of the catheter protruded outside from the cap through the aperture.

7. The catheter insertion assist device according to claim 6, further comprising:
sealing member arranged around the entire perimeter of the aperture of the cap, and for sealing in a liquid-tight manner a space between the ultrasonic element and an affected area including a region irradiated with an ultrasonic wave from the ultrasonic element; and
liquid supplying device for filling the space with a liquid.

8. The catheter insertion assist device according to claim 7 further comprising: liquid circulating device for circulating the liquid in the space.

9. The catheter insertion assist device according to claim 1, further comprising: monitoring element for monitoring a protrusion of the catheter.

10. The catheter insertion assist device according to claim 9, wherein the monitoring element is a monitoring ultrasonic element acquiring an ultrasonic tomogram in the proximity of the protrusion position of the catheter.

11. A treatment apparatus, comprising: the catheter insertion assist device according to claim 1 and a catheter, and
further comprising a potential detecting electrode detecting a potential of body tissue that is in contact with a tip of the catheter.

12. The catheter insertion assist device according to claim 6, further comprising: monitoring element for monitoring a protrusion of the catheter.

13. The catheter insertion assist device according to claim 12, wherein the monitoring element is a monitoring ultrasonic element acquiring an ultrasonic tomogram in the proximity of the protrusion position of the catheter.

14. A treatment apparatus, comprising: the catheter insertion assist device according to claim 6 and a catheter, and
further comprising a potential detecting electrode detecting a potential of body tissue that is in contact with a tip of the catheter.
